(19) United States
(12) Patent Application Publication
(10) Pub. No.: US 2021/0084905 A1
(43) Pub. Date: Mar. 25, 2021

(54) METHODS AND COMPOSITIONS FOR AQUACULTURE

(71) Applicant: President and Fellows of Harvard College, Cambridge, MA (US)

(72) Inventors: John Mekalanos, Brighton, MA (US); William Robbins, Boston, MA (US)

(21) Appl. No.: 16/970,848

(22) PCT Filed: Feb. 19, 2019
(86) PCT No.: PCT/US2019/018563

§ 371 Date: Aug. 18, 2020

Related U.S. Application Data
(60) Provisional application No. 62/632,085, filed on Feb. 19, 2018.
(60) Provisional application No. 62/632,729, filed on Feb. 20, 2018.

(51) Int. Cl. A01N 63/20; A01K 61/59

(52) U.S. Cl. CPC A01N 63/20; A01K 61/59

(57) ABSTRACT

In certain aspects, disclosed herein are novel compositions and methods related to aquaculture.

### RELATED U.S. APPLICATIONS

This application claims priority to U.S. Provisional Application 62/632,085 filed Feb. 19, 2018, and U.S. Provisional Application 62/632,729, filed Feb. 20, 2018, each of which is incorporated herein by reference in its entirety

### BACKGROUND

Acute hepatopancreatic necrosis syndrome (AHPNS) or early mortality syndrome (EMS) is an emerging epizootic disease that impacts Litopenaeus vannamei shrimp. The causative agent is Vibrio bacteria that harbor a plasmid that encodes a secreted binary toxin (PirAB) that possesses structural similarity to other known insecticidal toxins. The current disease model proposes V. parahaemolyticus strains that carry this vector colonize shrimp and then secrete toxin molecules causing necrosis of the shrimp hepatopancreatic tissue thus leading to mortality. Because V. parahaemolyticus is prevalent and ubiquitous in warm seawater and brackish estuaries and also found to colonize crustaceans and other invertebrates, it has been nearly impossible to completely protect large scale aquaculture from AHPNS strains where shrimp are farmed. AHPNS was first identified in China in 2009 and spread to Malaysia, Vietnam, Thailand, and the Philippines through 2013 and remains a problem. In 2017 it was also detected in shrimp aquaculture in Latin America. The Global Aquaculture Alliance (GAA) estimated losses caused by EMS disease to exceed one billion dollars per year. Thus, there is a need for novel and innovative treatments for AHPNS in aquaculture.

### SUMMARY

Provided herein are compositions and methods of preventing or treating acute hepatopancreatic necrosis syndrome (AHPNS) or early mortality syndrome (EMS) in cultured (e.g., farmed) crustaceans (e.g., shrimp or prawns).

The methods provided herein comprise methods of inhibiting the growth of pathogenic bacteria in cultured crustaceans by administering Aeromonas hydrophila A603 bacteria to environmental waters comprising the cultured crustaceans. The pathogenic bacteria may be Vibrio bacteria (e.g., V. cholera, V. vulnificus, V. harveyi, V. cholerae, V. aliginolyticus. A. hydrophila, or A. media). The Vibrio bacteria may be V. parahaemolyticus. The pathogenic bacteria may be an Aeromonas pathogenic bacteria (i.e., not Aeromonas hydrophila A603), such as A. hydrophila, A. caviae, A. sobria, or A. media. The bacteria may be antibiotic resistant. The pathogenic bacteria may be any pathogenic bacteria, including, but not limited to, the bacteria disclosed herein or any bacteria found in or on the bodies of shrimp. The pathogenic bacteria may be associated with acute hepatopancreatic necrosis syndrome (AHPNS) or early mortality syndrome (EMS). The pathogenic bacteria may not associated with acute hepatopancreatic necrosis syndrome (AHPNS) or early mortality syndrome (EMS).

Also provided herein are methods of treating or preventing bacterial infection in cultured crustaceans by administering Aeromonas hydrophila A603 bacteria to environmental waters comprising the cultured crustaceans. The bacterial infection may be caused by Vibrio bacteria (e.g., V. cholera, V. vulnificus, V. harveyi, V. cholerae, V. aliginolyticus. A. hydrophila, V. parahaemolyticus or A. media). The bacterial infection may be caused by an Aeromonas pathogenic bacteria (i.e, not Aeromonas hydrophila A603) such as A. hydrophila, A. caviae, A. sobria, or A. media. The bacterial infection is caused by pathogenic bacteria that are antibiotic resistant. The bacterial infection may be caused by any pathogenic bacteria, including, but not limited to, the pathogenic bacteria disclosed herein or any bacteria found in or on the bodies of shrimp. In some embodiments, the pathogenic bacteria causing the bacterial infection is associated with acute hepatopancreatic necrosis syndrome (AHPNS) or early mortality syndrome (EMS). In other embodiments, the pathogenic bacteria causing the bacterial infection is not associated with acute hepatopancreatic necrosis syndrome (AHPNS) or early mortality syndrome (EMS). In some aspects, provided herein are methods of overcoming or inhibiting antibiotic resistance in cultured crustaceans comprising administering Aeromonas hydrophila A603 bacteria to environmental waters comprising the cultured crustaceans.

The methods include treating or preventing bacterial (e.g., Vibrio bacteria) infection in cultured crustaceans by administering the Aeromonas hydrophila A603 bacteria to the environmental waters comprising the cultured crustaceans. In some aspects, provided herein are methods of inhibiting the growth of Vibrio bacteria in cultured crustaceans comprising administering Aeromonas hydrophila A603 bacteria to environmental waters comprising the cultured crustaceans. In some aspects, provided herein are methods of treating or preventing acute hepatopancreatic necrosis syndrome (AHPNS) or early mortality syndrome (EMS) in cultured crustaceans comprising administering Aeromonas hydrophila A603 bacteria to environmental waters comprising the cultured crustaceans. In some embodiments, the crustaceans are in a larval stage. In some embodiments, the crustaceans are mature crustaceans. The cultured crustaceans may be shrimp (e.g., Litopenaeus vannamei shrimp). In some embodiments, the environmental waters have tested positive for Vibrio bacteria or any bacteria causing acute hepatopancreatic necrosis syndrome (AHPNS) or early mortality syndrome (EMS). In some embodiments, the environmental waters have tested positive for Vibrio bacteria, and the Vibrio bacteria are antibiotic resistant. The Vibrio bacteria may be V. cholera or V. parahaemolyticus (e.g., EMS V. parahaemolyticus). The environmental waters may be seawater or brackish water. In some embodiments, the Vibrio bacteria is associated with acute hepatopancreatic necrosis syndrome (AHPNS) or early mortality syndrome (EMS). In other embodiments, the Vibrio bacteria is not associated with acute hepatopancreatic necrosis syndrome (AHPNS) or early mortality syndrome (EMS).

In some embodiments, the method further comprises contacting the Aeromonas hydrophila A603 bacteria with an agent that increases the expression of T6SS proteins. In some embodiments, the method further comprises contacting the Aeromonas hydrophila A603 bacteria with an agent that increases the expression of T6SS proteins prior to administering the Aeromonas hydrophila A603 bacteria to the environmental waters comprising the cultured crustaceans. In some embodiments, the method further comprises contacting the Aeromonas hydrophila A603 bacteria with an agent that increases the expression of T6SS proteins by administering the Aeromonas hydrophila A603 bacteria and the agent to the environmental waters comprising the cultured crustaceans. The agent may be an expression vector. In some embodiments, the expression vector encodes for a T6SS effector protein or a T6SS machinery protein (e.g., a protein that aids in or is part of T6SS assembly).

In some embodiments, the method further comprises contacting the Aeromonas hydrophila A603 bacteria with an agent that activates phenazine biosynthesis. In some embodiments, the method further comprises contacting the Aeromonas hydrophila A603 bacteria with an agent that activates phenazine biosynthesis prior to administering the Aeromonas hydrophila A603 bacteria to the environmental waters comprising the cultured crustaceans. The agent may be an expression vector. In some embodiments, the expression vector comprises a gene or a portion of a gene in the phenazine operon. The agent may be an acylated homoserine lactone (AHL) molecule or a PhzR protein.

In some embodiments, the method further comprises administering the Aeromonas hydrophila A603 bacteria to the environmental waters comprising the cultured crustaceans conjointly with a phenazine (e.g., pyocyanin), a phenazine precursor, or phenazine derivative.

In some aspects, provided herein are methods of treating a bacterial growth or infection (e.g., a bacterial growth or infection described herein) by administering phenazine (e.g., pyocyanin), a phenazine precursor, or phenazine derivative to the environmental waters comprising the cultured crustaceans. In some embodiments, the methods comprise administering phenazine (e.g., pyocyanin), a phenazine precursor, or phenazine derivative to the environmental waters comprising the cultured crustaceans.

In some embodiments, the Aeromonas hydrophila A603 is administered to the environmental waters comprising the cultured crustaceans conjointly with an antibiotic. The antibiotic may be administered to the environmental waters comprising the cultured crustaceans prior to Aeromonas hydrophila A603 administration. The antibiotic (e.g., an antibiotic that does not target Aeromonas hydrophila A603) may be administered to the environmental waters comprising the cultured crustaceans simultaneous to Aeromonas hydrophila A603 administration.

Provided herein are compositions comprising Aeromonas hydrophila A603. The composition may be a probiotic. The composition may be feed used for industrial crustaceans farming or aquaculture. The compositions disclosed herein may comprise a phenazine (e.g., pyocyanin). The composition may comprise any agent that increases phenazine biosynthesis (e.g., an AHL molecule or a PhzR protein). In some embodiments, the composition comprises a T6SS effector protein. The composition may further comprise and antibiotic (e.g., an antibiotic that does not target Aeromonas hydrophila A603).

### BRIEF DESCRIPTION OF THE DRAWINGS

FIG. 1A shows the phylogeny of EMS and non-EMS strains using 1269 conserved core genes

FIG. 1B shows the phylogeny of EMS PirA/B plasmid using complete sequence of virulence plasmid.

FIG. 2 has three parts, A-C, and shows identification of an Aeromonad with antibacterial activity against bacteria of the genus Vibrio was identified in a screen using a panel of bacterial isolates from shrimp being sold in a Boston area seafood market that were imported from China (Part C). Shrimp-isolated Aeromonas (A603 and A604) killed Vibrio sp. Strains C6706 (Part B) and A608 (Part C) when co-cultured for 3 hours on LB agar

FIG. 3 shows the design of a screen to incubate both a candidate “predator” strain and a Vibrio “prey” strain together and a T6SS contact-dependent killing assay. The steps are as follows: 1) Plate˜100 million bacteria as both a monoculture and mixture in <10 ul on an agar surface and allow to excess liquid to dry as a high-density culture. 2) Incubate 2-3 hours at 37 C. 3) Re-suspend cells and make serial dilutions. 4) Plate on selective media, grow O/N, and enumerate survival of selected strains.

FIG. 4 shows 100 million Vibrio incubated with Aeromonas A603 for two hours and panel aquatic and shrimp isolated bacteria co-incubated with A603.

FIG. 5 has two parts, A and B, and shows the role of VipA/VipB in T6SS protein ejection and diagram of vipA gene deletion in A603 vipA strain. Part A shows deletion of vipA gene. Part B shows T6SS apparatus requires structural VipA/VipB proteins to function.

FIG. 6A shows that Aeromonas A603 secretes a diffusible antibacterial molecule and that secreted molecule is T6SS− independent.

FIG. 6B shows the transposon insertion in Aeromonas A603 vipA abolishes killing activity using secretion assay.

FIG. 7 shows that a phenazine-based molecule has broad spectrum antibacterial activity.

FIG. 8 has four parts, A-D, and shows T6SS and phenazine operons in A603. Part A shows A603 T6SS core genetic operon (T6SS Island I). Part B shows A603 T6SS accessory operon (T6SS Island II). Part C shows A603 phenazine biosynthetic operon (arrows are transpososon insertions that abolished activity). Part D shows Predicted quorum sensing-related crosstalk and positive feedback of genetic elements in both Phenazine Biosynthetic Operon and T6SS operon. Typically phzI/phzR gene are found together in other phenazineoperons (Erwinia, Pseudomonads). PhzI protein is an Autoinducer Synthase (AI-2). PhzR protein is an AI-2 regulated Transcriptional Regulator. Crosstalk between T6SS and phenazinemay help explain the synergistic (more than additive) antibiotic effects of both observed using single and double mutants against some bacterial strains.

FIG. 9 shows the testing the contribution of both or either T6SS and phenazine for killing EMS strain of Ta Mai. Specifically, the testing comprised the steps of creating a 1:1 Mixture A603 and Ta Mai (EMS V. parahaemolyticus strain)(except for no challenge, EMS alone), and a 2 Hour 37 C incubation on agar (˜100 million of each bacteria). The mixture was resuspended, mixed, and plated on selective media.

FIG. 10 shows A603 phenazine operon and conserved predicted synthesis pathway.

FIG. 11 has two parts, A-B. Part A shows A603 phenazine operon and predicted functions of various genes. Part B shows known phenazines molecules with side groups that may resemble the A603 modified phenazine molecule.

FIG. 12 has two parts, A and B. Part A shows infection of shrimp with A603 and Vibrio. Part B shows normalized Illumina reads to measure A603 and EMS strain abundance in shrimp.

FIG. 13 has three parts, A-C, and shows that adding Aeromonas A603 to shrimp reduced the species diversity slightly to between 38 and 80 per shrimp with a significant reduction of both environmental (B1 & B2) and environmental EMS Vibrio (B3 & B4) while Aeromonas was calculated to comprise only ˜2% of the microbiome. Part A shows the percentage microbiome that belongs to Vibrio. Part B shows the percentage microbiome that belongs to Aeromonas. Part C the calculated species diversity (alpha-diversity).

FIG. 14 shows the role of T6SS in protection, shrimp were infected with EMS strain Ta Mai with and without pre-inoculation of strains A603 and A603 vipA. EMS strain (Ta Mai) is less abundant in shrimp challenged with A603. A603 T6SS+ more effective than A603 T6SS− (vipA) at protection. Colonization of Isolate B is inversely correlated with colonization by Ta Mai.

FIG. 15 shows the predicted phylogenetic change in the microbiome when treated with A603 for 24 hours). 21 most abundant bacterial families represented in shrimp microbiome 24 hours post-treatment using A603. Deep sequence Illumina data was analyze by MG-RAST. Families exhibiting most increased and decreased abundance are highlighted.

FIG. 16 shows the implementation of methods disclosed herein in aquaculture. A603 can be added at first step to colonize small/growing shrimp in a controlled environment. Smaller volume is needed to infect shrimp by adding to water. One small bacterial culture grown to high density in a flask sufficient is for inoculation. This provides protection before shrimp are moved to ponds where (re)infection by EMS strains is a more significant problem. Shrimp transitioned to pond bring A603 into environment. A naturally selected streptomycin resistance marker in the A603 strain (or PCR) makes the detection and measurement of A603 in shrimp/water easy. A603 has very fast doubling time and can be added to ponds or with feed if necessary. A small bioreactor can readily grow 10 billion cfu/ml (10 trillion cfu/L) in a day with basic bacterial media rendering inoculation of ponds very efficient. A 50 million gallon pond, which is equivalent to about 75 Olympic swimming pools, can be inoculated with 10,000-100,000 cfus/ml using one 5 L bioreactor and very economical bacterial media (˜$50 for 5 L). This inoculum is similar to that used in protection assays in the lab (10 cfu/ml).

FIG. 17 shows plots illustrating DNA and RNA abundance using data from next-generation sequencing (NGS) and mapping of either bacterial DNA and RNA from uninfected and infected shrimp. Each plot point represents a relative abundance of reads mapped to a bacterial family for each group. There are four shrimp per group and all reads were at least 100 nucleotides in length and mapped using Metagenomic Rapid Annotations using Subsystems Technology (MG-RAST).

FIG. 18 shows transcriptome (RNA sequencing) of the shrimp body is the best indicator of the increased bacterial load of Vibrionaceae.

FIG. 19 shows shrimp pretreated with A603 have 3-fold fewer Vibrionaceae and 8-fold fewer Aeromonadaceae after 72 hours. environmental Vibrionaceae and 8-fold fewer Aeromonadaceae after 72 hours. Shrimp pretreated with A603 and then infected have 10-fold less Vibrionaceae (environmental and AHPND) and 97-fold less Aeromonadaceae after 72 hours than uninfected.

FIG. 20 shows the antibiotic contribution of both T6SS and phenazine for sensitive strains. Bacteria vary in their sensitivity to T6SS and phenazine. Specifically, T6SS is dominant, phenazine-like molecule appears to be additive or even synergistic when bactericidal, and co-regulation suggests these work together.

FIG. 21 shows that the A603 transcriptome vs Ta Mai transcriptome co-cultured.

FIG. 22 shows highly upregulated genes in Ta Mai: Ox-Redox, Electron transfer, Glyoxalase-like found in the same operons.

FIG. 23 shows EphR-like proteins confer phenazine resistance when expressed in sensitive strains.

FIG. 24 shows that monooxygenases does not confer phenazine resistance in V. cholerae—but does steal Fe(II)—which can be used by EphR.

FIG. 25 shows that A603 may work also as an antibacterial in the environment or host.

FIG. 26 shows quantification of protection in shrimp using NGS sequencing.

FIG. 27 shows that A603-treated shrimp have reduced colonization, not only by added EMS strains but also by endemic, environmental Vibrio strains.

### DETAILED DESCRIPTION

### General

Provided herein are methods and compositions of aquaculture. Such methods include preventing or treating acute hepatopancreatic necrosis syndrome (AHPNS) or early mortality syndrome (EMS) in cultured crustaceans (e.g., shrimp or prawns). The methods include treating or preventing bacterial (e.g., Vibrio bacteria) infection in cultured crustaceans by administering Aeromonas A603 bacteria to the environmental waters comprising the cultured crustaceans.

### Definitions

For convenience, certain terms employed in the specification, examples, and appended claims are collected here.

As used herein, the term “administering” means providing an agent or composition to the environmental waters comprising crustaceans described herein. “Administering” may include any other means of providing the crustaceans described herein with the compositions described herein.

The term “agent” is used herein to denote a chemical compound, a small molecule, a mixture of chemical compounds and/or a biological macromolecule (such as a small molecule or a protein or a peptide). The activity of such agents may render them suitable as a “agent” which is a biologically, physiologically, or pharmacologically active substance (or substances) that acts locally or systemically.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

The term “peptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

As used herein, the term “subject” means a non-human animal selected for treatment or therapy, such as shrimp. The term “shrimp” includes decapod crustaceans. Used broadly, it may cover any of the groups with elongated bodies and a primarily swimming mode of locomotion, such groups include Caridea and Dendrobranchiata.

“Treating” a disease in a subject or “treating” a subject having a disease refers to subjecting the subject to a pharmaceutical treatment, e.g., the administration of a drug, such that at least one symptom of the disease is decreased or prevented from worsening.

### Methods

Provided herein are methods of preventing or treating acute hepatopancreatic necrosis syndrome (AHPNS) or early mortality syndrome (EMS) in cultured crustaceans (e.g., shrimp or prawns). Provided herein are methods of treating or preventing pathogenic bacterial (e.g., Vibrio bacteria or pathogenic Aeromonas bacteria, not including Aeromonas hydrophila A603 bacteria) infection or inhibiting pathogenic bacterial growth by administering Aeromonas hydrophila A603 bacteria or compositions comprising Aeromonas hydrophila A603 bacteria to the environmental waters comprising the cultured crustaceans. The methods provided herein comprise methods of inhibiting the growth of pathogenic bacteria in cultured crustaceans by administering Aeromonas hydrophila A603 bacteria to environmental waters comprising the cultured crustaceans. The pathogenic bacteria may be any species of Vibrio bacteria (e.g., V. cholera, V. vulnificus, V. harveyi, V. cholerae, V. aliginolyticus. A. hydrophila, or A. media). The Vibrio bacteria may be V. parahaemolyticus. The pathogenic bacteria may be an Aeromonas pathogenic bacteria (i.e., not Aeromonas hydrophila A603), such A. hydrophila or A. media. The bacteria may be antibiotic resistant. The pathogenic bacteria may be any pathogenic bacteria, including, but not limited to, the bacteria disclosed herein or any bacteria found in or on the bodies of shrimp. The pathogenic bacteria may be associated with acute hepatopancreatic necrosis syndrome (AHPNS) or early mortality syndrome (EMS). The pathogenic bacteria may not associated with acute hepatopancreatic necrosis syndrome (AHPNS) or early mortality syndrome (EMS).

Also provided herein are methods of treating or preventing bacterial infection in cultured crustaceans by administering Aeromonas hydrophila A603 bacteria to environmental waters comprising the cultured crustaceans. The bacterial infection may be caused by Vibrio bacteria (e.g., V. cholera, V. vulnificus, V. harveyi, V. cholerae, V. aliginolyticus. A. hydrophila, V. parahaemolyticus or A. media). The bacterial infection may be caused by an Aeromonas pathogenic bacteria (i.e, not Aeromonas hydrophila A603) such as A. hydrophila or A. media. The bacterial infection is caused by pathogenic bacteria that are antibiotic resistant. The bacterial infection may be caused by any pathogenic bacteria, including, but not limited to, the pathogenic bacteria disclosed herein or any bacteria found in or on the bodies of shrimp. In some embodiments, the pathogenic bacteria causing the bacterial infection is associated with acute hepatopancreatic necrosis syndrome (AHPNS) or early mortality syndrome (EMS). In other embodiments, the pathogenic bacteria causing the bacterial infection is not associated with acute hepatopancreatic necrosis syndrome (AHPNS) or early mortality syndrome (EMS). In some aspects, provided herein are methods of overcoming or inhibiting antibiotic resistance in cultured crustaceans comprising administering Aeromonas hydrophila A603 bacteria to environmental waters comprising the cultured crustaceans.

The crustaceans described herein may be any crustacean cultured in industrial aquaculture. Examples of such crustaceans, include, but are not limited to, shrimp of the family Penaeidae, such as Litopenaeus vannamei (Pacific white shrimp) and Penaeus monodon (giant tiger prawn). Administering Aeromonas hydrophila A603 bacteria or the compositions described herein may be administered at any point in the crustaceans life cycle, including egg, larval, nauplii, mysis, postlarvae, and/or mature (adult) stages. In some embodiments, the crustaceans are mature crustaceans. In some embodiments, the environmental waters have tested positive for Vibrio bacteria and/or any bacteria causing acute hepatopancreatic necrosis syndrome (AHPNS) or early mortality syndrome (EMS). In some embodiments, the environmental waters have tested positive for Vibrio bacteria, and the Vibrio bacteria are antibiotic resistant. The Vibrio bacteria may be V. cholera or V. parahaemolyticus (e.g., EMS V. parahaemolyticus). In some embodiments, the Vibrio bacteria may be a Ta Mai strain of Vibrio bacteria. Environmental waters, as described herein, may be any body of water comprising crustaceans. The environmental waters may be seawater, brackish water, or the water may comprise freshwater.

In some embodiments, the method further comprises contacting the Aeromonas hydrophila A603 bacteria with an agent (e.g., a nucleic acid vector) that increases the expression of type VI secretion system (T6SS) proteins. The T6SS is molecular machine used by a wide range of gram-negative bacterial species to transport proteins from the interior (cytoplasm or cytosol) of a bacterial cell across the cellular envelope into an adjacent target cell. The T6SS consists of proteins that assemble into three sub-complexes: a phage tail-like tubule, a phage baseplate-like structure, and cell-envelope spanning membrane complex. These three subcomplexes work together to transport proteins across the bacterial cell envelope and into a target cell through a contractile mechanism. In some embodiments, the expression vector encodes proteins involved in T6SS machinery or assembly, such as proteins that assemble the phage tail-like tubule, a phage baseplate-like structure, and/or the cell-envelope spanning membrane complex. In some embodiments, the expression vector may encode for T6SS effector proteins, such as toxic proteins delivered by T6SS machinery to the target bacteria. Examples of T6SS effector proteins include, but are not limited to, proteins encoded by tseA, tseB, and tseC (e.g., TseA, TseB, and TseC). T6SS effector proteins may include any T6SS effector protein that are antibacterial by function.

Provided herein are nucleic acid molecules or polynucleotides that encode the T6SS proteins described herein. For example, the polynucleotide may encode a T6SS protein or fragment thereof. The nucleic acids may be present, for example, in whole cells, in a cell lysate, or in a partially purified or substantially pure form.

Nucleic acids described herein can be obtained using standard molecular biology techniques. For example, nucleic acid molecules described herein can be cloned using standard PCR techniques or chemically synthesized.

In certain embodiments, provided herein are vectors that contain the isolated nucleic acid molecules described herein (e.g., a T6SS protein). As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

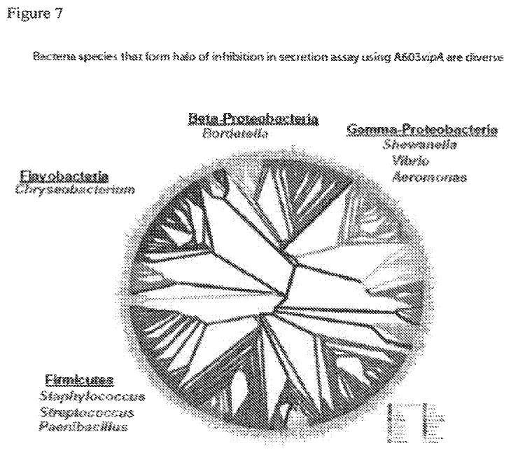

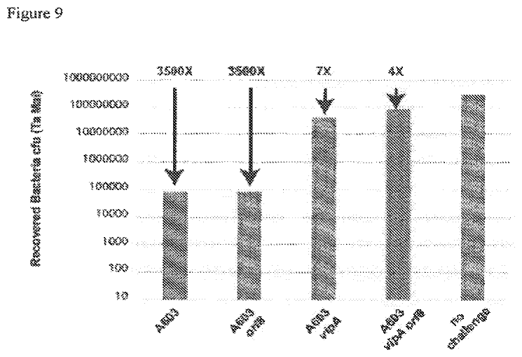

In some embodiments, the method further comprises contacting the Aeromonas hydrophila A603 bacteria with an agent (e.g., a nucleic acid vector, a peptide, or a small molecule) that activates phenazine biosynthetic pathway. The agent may be an acylated homoserine lactone (AHL) or a PhzR protein. The agent may be an expression vector. In some embodiments, the expression vector may comprise a gene or part of a gene in the phenazine biosynthetic operon. Examples of such genes include, but are not limited to, genes listed in FIG. 8C. The agent may be a peptide. The peptide may be, for example, a phenazine precursor or any other compound in the phenazine biosynthetic pathway. In some embodiments, the method further comprises administering the Aeromonas hydrophila A603 bacteria to the environmental waters comprising the cultured crustaceans conjointly with a phenazine, a phenazine precursor, a phenazine derivative, a phenazine based molecule, or any other intermediate molecule in the phenazine biosynthetic pathway. The phenazine may be pyocyanin. Examples of phenazine precursors, derivatives, or intermediates may be found in FIG. 10. More details on phenazine products can be found in Laursen J B, Nielsen J. Phenazine natural products: Biosynthesis, synthetic analogues, and biological activity. Chemical Reviews. 2004; 104: 1663-1685, hereby incorporated in its entirety.

Antibiotics are commonly used in shrimp farming to prevent or treat disease outbreaks. In some embodiments, Aeromonas hydrophila A603 bacteria is administered to the environmental water comprising the cultured crustaceans conjointly with one or more antibiotics. The antibiotic may be administered to the environmental waters comprising the cultured crustaceans prior to Aeromonas hydrophila A603 administration, in order to substantially eliminate existing bacteria in the cultured crustaceans and/or the environmental waters. For example, antibiotics may be administered to the environmental waters, and, after a period of time, Aeromonas hydrophila A603 is administered to the environmental waters. A period of time may be at least an hour, at least 24 hours, at least 48 hours, at least one week, at least two weeks, or at least a month. The one or more antibiotics may be selected from oxytetracycline, florfenicol, sarafloxacin, enrofloxacin, chlortetracycline, quinolones, ciprofloxacin, norfloxacin, oxolinic acid, perfloxacin, sulfamethazine, gentamicin, and/or tiamulin. The antibiotic (e.g., an antibiotic that does not target Aeromonas hydrophila A603) may be administered to the environmental waters comprising the cultured crustaceans simultaneously to Aeromonas hydrophila A603 administration. Aeromonas hydrophila A603 may be antibiotic resistant. The antibiotic (e.g., streptomycin) may be administered to the environmental waters comprising the cultured crustaceans simultaneously to antibiotic resistant (e.g., streptomycin-resistant) Aeromonas hydrophila A603 administration. In certain embodiments, agents of the invention may be used alone or conjointly administered with another type of therapeutic agent (e.g., an antibiotic). As used herein, the phrase “conjoint administration” refers to any form of administration of two or more different therapeutic agents/bacteria such that the second agent is administered while the previously administered therapeutic agent is still effective in the body or environmental waters (e.g., the two agents are simultaneously effective in the subject, which may include synergistic effects of the two agents). For example, the different therapeutic agents/bacteria can be administered either in the same formulation or in separate formulations, either concomitantly or sequentially.

Aeromonas hydrophila A603 may be administered at any concentration or dosage needed to inhibit pathological bacteria (e.g., Vibrio bacteria) growth or treat or prevent bacterial infection. Aeromonas hydrophila A603 may be measured in colony forming units (cfu). Aeromonas hydrophila A603 may be administered in approximately equal, less, or more colony forming units as the estimated amount of pathological bacteria in the environmental waters. For example, at least 1 million, at least 100 million, at least 500 million, at least 1 billion, at least 100 billion, at least 500 billion, at least 1 trillion, at least 100 trillion, at least 500 trillion, at least 1 quadrillion, at least 100 quadrillion, or at least 500 quadrillion may be administered to the environmental waters comprising the cultured crustaceans.

### Compositions

Provided herein are compositions that comprise Aeromonas hydrophila A603 for the treatment or prevention of acute hepatopancreatic necrosis syndrome (AHPNS) or early mortality syndrome (EMS) in cultured crustaceans (e.g., shrimp or prawns). In some embodiments, the composition is a probiotic. Compositions disclosed herein may be used as a feed additive or supplement in crustacean farming techniques. For example, the compositions disclosed herein may be added to processed fish meal prior to feeding crustaceans in aquaculture. The feed maybe a dry feed. The feed may be a micro-encapsulated feed. The feed may be entrapped in a liposome and the liposome is further encapsulated in a hydrocolloid matrix. The feed may be in the form of complex microcapsules (CXMs) consisting of dietary ingredients and lipid-wall microcapsules (LWMs) embedded in particles of a gelled mixture of alginate and gelatin to obtain a single food-particle type used to provide suspension feeders with dietary nutrients. The feed may be a liquid food stuff. Liquid foodstuff may include particulate feed in a liquid medium and provides an easy convenient way to deliver a nutritionally formulated ration to crustaceans. The liquid foodstuff may include oil-coated nutrient feed particles which are embedded in a gel or a food in a polymer blend. The gel may be crosslinked or complexed to encapsulate the oil-coated nutrient to provide encapsulated oil-coated nutrient feed. The particulate feed may be adjusted for the requirements of the marine animal being fed. The feed may comprise animal protein, brine shrimp, egg product, betaine, alanine, isoleucine, leucine, serine, valine, glycine, astaxanthin, vitamin A supplement, vitamin B 12 supplement, riboflavin supplement, calcium pantothenate, niacin supplement, vitamin D 3 supplement, vitamin E supplement, menadione sodium bisulfite complex, folic acid, biotin, thiamine, pyridoxine hydrochloride, inositol and/or choline chloride.

In some embodiments, the composition further comprises a phenazine. In some embodiments, the composition further comprises an activator of the phenazine biosynthetic pathway (e.g., AHL or a PhzR protein). In some embodiments, the composition further comprises an antibiotic. The antibiotic may be oxytetracycline, florfenicol, sarafloxacin, enrofloxacin, chlortetracycline, quinolones, ciprofloxacin, norfloxacin, oxolinic acid, perfloxacin, sulfamethazine, gentamicin, or tiamulin. In some embodiments, the composition further comprises a phenazine, a phenazine precursor, a phenazine derivative, a phenazine based molecule, or any other intermediate molecule in the phenazine biosynthetic pathway. The phenazine may be pyocyanin. Examples of phenazine precursors, derivatives, or intermediates may be found in FIG. 10. More details on phenazine products can be found in Laursen J B, Nielsen J. Phenazine natural products: Biosynthesis, synthetic analogues, and biological activity. Chemical Reviews. 2004; 104: 1663-1685, hereby incorporated in its entirety.

### EXEMPLIFICATION

### Example 1: Isolation of Probiotic Strain and Characterization of In Vitro Antibacterial Activity

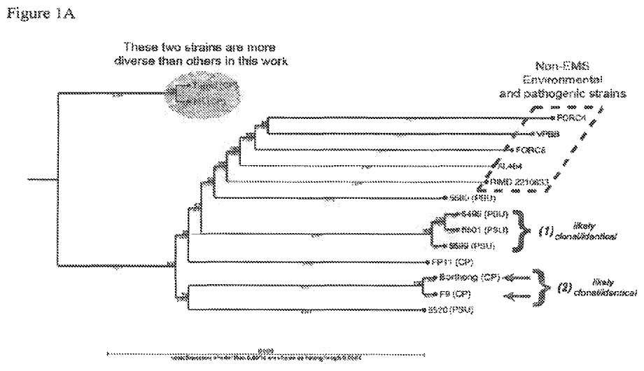

Strains isolated from infected shrimp that also carry the genes for the toxin on a plasmid are not always clonal or closely related suggesting the transmission of this plasmid among environmental Vibrio parahaemolyticus strains is sufficient to create a diverse assemblage of pathogenic strains and that the disease may be solely attributed to this vector (FIGS. 1A and 1B). Other species of Vibrio carrying the PirAB toxin plasmid have now been isolated from diseased shrimp. Based this observation, an antibiotic or bacteriophage-based therapeutic that targets and kills a specific EMS strain may not be efficient against another due to innate differences in strains. A probiotic strain that can cohabitate with shrimp and that can kill a broad spectrum of Vibrio strains without selecting for resistance was a key aim that initiated this work.

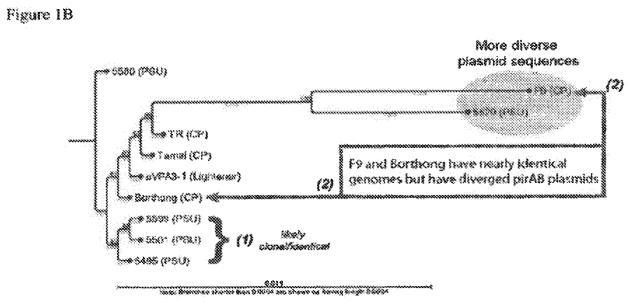

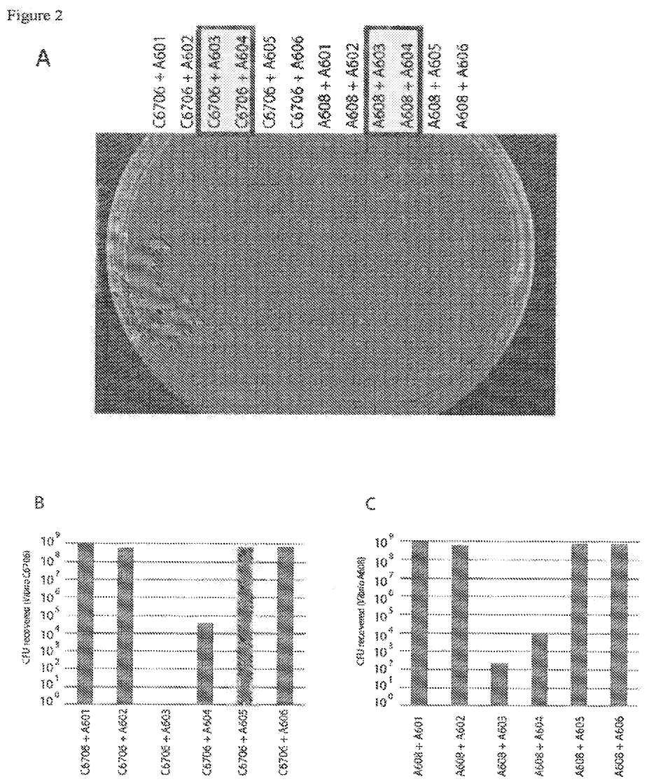

An Aeromonad with antibacterial activity against bacteria of the genus Vibrio was identified in a screen using a panel of bacterial isolates from shrimp imported from China (FIG. 2). This candidate strain (designated Aeromonas hydrophila A603) was found to have the most significant antibacterial activity of strains tested. The design of this screen was to incubate both a candidate “predator” strain and a Vibrio “prey” strain together as a mixed high density co-culture on a semi-solid 1.6% agar surface for three hours and then re-suspend, mix, dilute, and plate on selective media in order to determine antibiotic activity against the prey strain by measuring the number of bacteria that cannot be recovered (FIG. 3). This screening assay can identify strains that kill using both contact-dependent and other mechanisms as a high percentage of the mixed bacterial cells are in are contact or close proximity to each other in an artificial biofilm-like condition and small inhibitory molecules can be cross-fed in the co-culture. This approach differs from traditional agar diffusion methods that are designed to identify small molecules that are secreted and also soluble enough to diffuse through an agar overlay and then inhibit the growth of an indicator or prey strain growing as a confluent lawn.

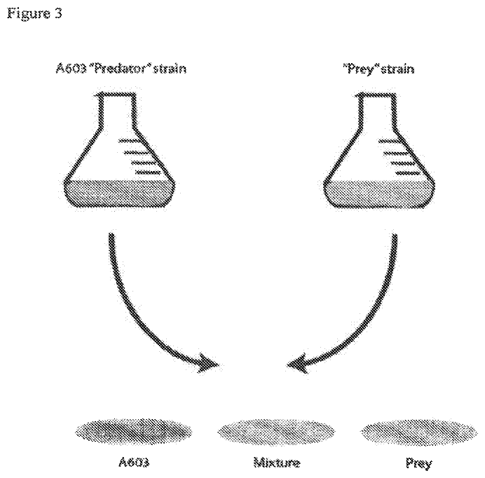

When approximately 100 million Vibrio are incubated with Aeromonas A603 for two hours and plated on selective media the result is that fewer than 100 colony forming units (cfu) of Vibrio can be recovered (FIG. 4). In contrast to Vibrio, there is an increase of A603 cfu measured. This indicates that nearly all Vibrio are killed while the viability of A603 is largely unaffected when the two strains are grown together. This significant killing activity is noted for all species, strains, and isolates of the genus Vibrio tested with A603. The host spectrum of antibacterial activity against both laboratory and environmental bacteria using the co-culture screen was found to be specific to the orders Vibrionales, Aeromonadales, and Alteromonadales (FIG. 4). Challenges against other laboratory bacteria including E. coli and clinical Pseudomonas strains showed insignificant activity as these strains are not killed by A603.

The genome of A603 was sequenced using both Illumina and Pacific Biosciences next generation sequencing platforms yielding a closed and complete 4.8-Megabase genome. Using the genome and predicted genes it was possible to identify potential antibacterial components in A603 by looking for the Type VI secretion system (T6SS), antimicrobial metabolic pathways, bacteriocins, and bacteriophages. In addition to a Mu phage, a T6SS genomic core island and a separate accessory T6SS island (Island II) are found in A603 and these resemble genetic operons found in other Aeromonas strains (FIG. 8A & FIG. 8B).

### Example 2: Characterization of A603 T6SS

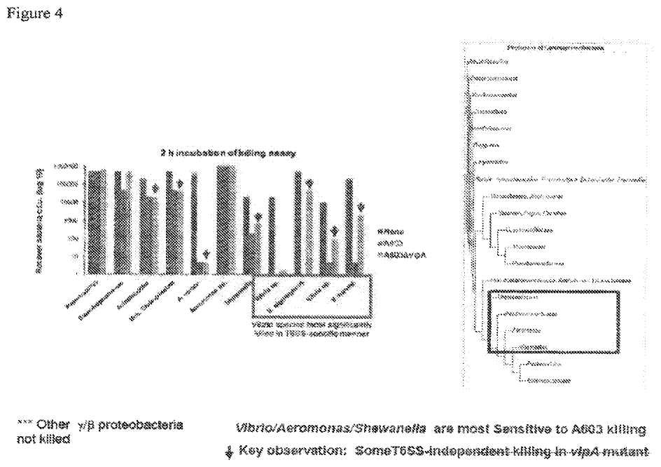

When the T6SS machinery is assembled, it targets other cells through a contact-dependent mechanism by contracting and then ejecting effector proteins across its membrane and into another “prey” cell (FIG. 5B). These effectors target the cell, bacterial or sometimes eukaryotic, often by forming pores or possessing an innate enzymatic activity such as one that degrades the cell wall or DNA. In A603, there are three predicted effectors (tseA, tseB, & tseC) positioned next to vrgG genes and all are predicted to be antibacterial by function. These effectors have different putative targets in the bacterial cell; TseA is similar to pore-forming colicins, TseB has a predicted nuclease domain and TseC is predicted to possess both peptidase and lysozyme activities. This strategy to hit multiple and diverse targets in a bacteria is a prevalent theme in other T6SSs and this may explain its general efficiency as an antibacterial mechanism. Bacteria that encode T6SS and effectors also encode cognate immunity proteins that both protect self and sister cells from T6SS when active.

To examine the role and contribution of the T6SS in A603 antibacterial activity, a gene that encodes a T6SS structural protein essential for activity was precisely deleted (FIG. 5A). This gene-encoded protein, VipA, is required for the T6SS apparatus to assemble, engage, and secrete antibacterial effectors (FIG. 5B). Both A603 and A603 ΔvipA were used to screen a panel of prey strains and determine the contributing role of T6SS antibacterial activity (FIG. 4). Absence of vipA significantly reduced, however did not abolish, killing for some bacteria suggesting a second T6SS-independent mechanism is also independently sufficient and most likely complements T6SS-mediated antibacterial activity.

### Example 3: Identification of Phenazine Operon as a Second Antibacterial Mechanism

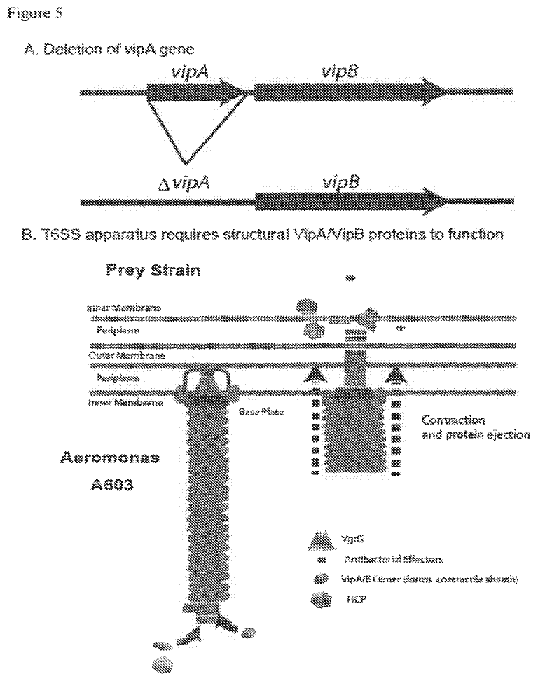

A603 inhibits the growth of a variety of bacterial strains seeded into an agar soft overlay by secretion of an antibacterial compound independent of the vipA gene (FIG. 6A). To identify this antibacterial mechanism, a transposon mutagenesis approach was used to screen for random genetic knockouts of A603 ΔvipA that then failed to kill a sensitive indicator strain when seeded in a soft agar overlay. Of 600 tested mutants, ˜12 insertions failed to kill in the secretion assay and the majority of insertions mapped to a predicted phenazine biosynthetic operon (arrows FIG. 8C). Many other phenazine molecules including pyocyanin are shown to elicit antibacterial activity often by causing oxidative stress. To test whether the genes hit in the transposon insertion screen are required, two separate genes with insertions (orf6 & orf8) in the region of the operon were precisely deleted in A603 ΔvipA. Both deleted genes were phenotypic to the corresponding insertions (FIG. 6B) and failed to form a halo of inhibition on seeded plates.

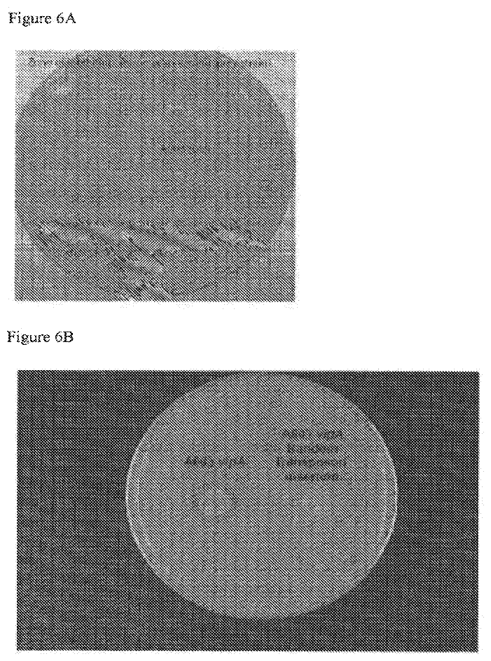

The spectrum of inhibition for the putative phenazine molecule was tested by seeding agar with a diverse set of laboratory and environmental strains using A603 ΔvipA and an isogenic double knockout A603 ΔvipA Δorf8 to score inhibition by appearance of a halo around A603 ΔvipA. Unlike T6SS, which is only known to target a subset of gram-negative bacteria, this secreted molecule inhibits the growth of a broad range of both gram negative and gram positive strains (FIG. 7).

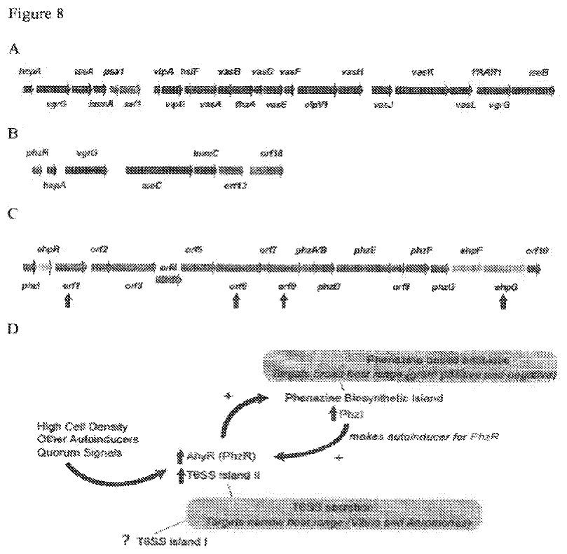

Both the T6SS and the phenazine-based molecule are shown to act independently as antimicrobial mechanisms when tested on Vibrio strains. A panel of A603 strains lacking either and both vipA and orf8 were incubated with Vibrio strains to measure the contribution of each antibacterial mechanism. When co-incubated with various Vibrio and Aeromonas strains, the sensitivity to each mechanism appears to vary in a bacteria-dependent manner and the A603 ΔvipA Δorf8 strain has no measurable antibacterial activity. For EMS strains, T6SS is the most significant killing mechanism within one hour as it's killing activity is ˜500× that of the phenazine (FIG. 9). It was concluded that these are the sole antimicrobial mechanisms in A603 against Vibrio and Aeromonas and that they act autonomously to inhibit or kill.

Synergistic cooperativity is not apparent or detected within the resolution of this assay however genetic elements within one T6SS cluster and the phenazine biosynthetic pathway suggest some co-regulation or crosstalk. The T6SS cluster II (FIG. 8B) includes a transcriptional activator that is very closely resembles the PhzR protein that activates phenazine synthesis in Pseudomonas. This activator includes a conserved domain that binds acylated homoserine lactone (AHL) molecules, a key component of quorum sensing. The phenazine pathway includes a gene that encodes PhzI, a protein that makes AHLs (FIG. 8C). This genetic circuit is predicted to be up-regulated at high bacterial density and the phenazine pathway would likely be subjected to positive feedback, possibly leading to very high expression of phenazine biosynthesis with T6SS (FIG. 8D). Another possibility is that Vibrio or other bacterial AHLs can co-activate this pathway in A603. This is currently being explored using RNAseq by comparing mono- and co-cultured A603 and mutants in either and both T6SS and phenazine biosynthesis. Engineering this operon to be more active is one goal to make this strain more potent when used as a targeted antibiotic treatment.

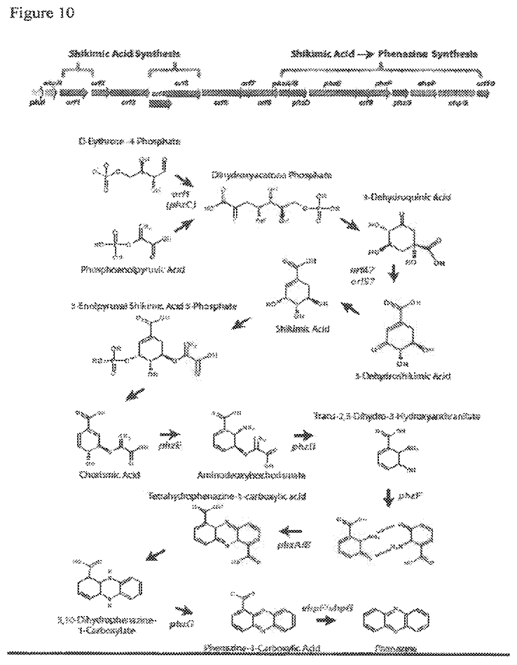

Some of the accessory or hypothetical genes in the phenazine operon are predicted to have functions critical to phenazine or shikimic acid synthesis while other genes present in the A603 genome appear unique and are not identified in other phenazine operons (FIG. 10 & FIG. 11). The gene orf2 is a SAM-dependent methyltransferase and orf6 is a D-alanyl ligase. Some known phenazine-based molecules are methylated and others modified with D-alanine and are indicated in FIG. 11B. D-alanylgriseoluteic acid is one example and it unique from many phenazine-based molecules as it uses a cognate immunity protein (ehpR) and a MFS efflux pump when secreted (9,10). The ehpR and a predicted MFS pump (orf3) are both present in A603 suggesting a similar mechanism and possible molecule. The remaining genes (orf7, orf8, orf9, & orf10) play an unknown role and may make additional modifications. An immediate goal is to purify this phenazine molecule and have its structure determined as it may be a novel molecule possessing additional properties or uses.

### Example 4: Identification of Phenazine Operon as a Second Antibacterial Mechanism

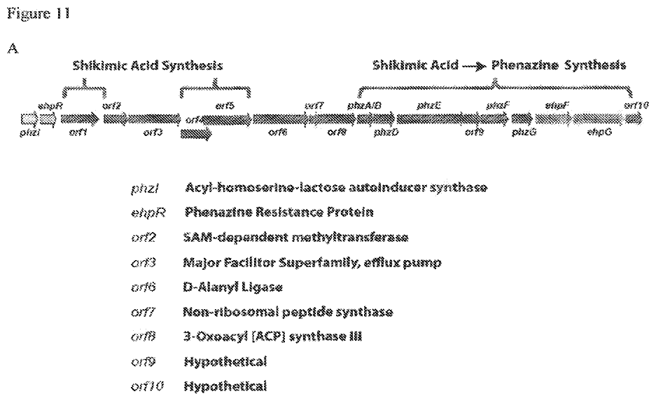

To test the antibiotic activity and a putative role in probiotic protection, shrimp (Litopenaeus vannamei) were incubated with Aeromonas A603 and EMS Vibrio parahaemolyticus strains to measure the colonization of each strain and whether A603 could antagonize colonization of EMS strains in vivo. This was done by adding 5×105/ml cfu of either or both strains to 500 ml seawater using 1-2 gram shrimp and then measuring bacteria in both water and shrimp over 48 hours. Bacterial abundance was measured using Illumina sequenced DNA obtained from bacteria isolated from 1 ml of water and from the filtrate of a homogenized shrimp. Upon DNA extraction, a quantified DNA standard for a ˜1000 bp nonbacterial artificial gene produced by PCR was also added to each sample before Illumina libraries were built and amplified in order to normalize reads as bacterial abundance could bias distributions of each strain. After Illumina sequencing, reads were mapped to the standard and also the A603 and EMS reference genomes including the pVPA3 PirA/B plasmid and the measure of each strain was calculated (FIG. 12).

Shrimp uninfected with EMS (24 hours+/−A603, A1/A2 & B1/B2) were found to have an apparent low level of colonization of EMS Vibrio (10-100 per shrimp) prior to infection with Ta Mai strain, but this is likely due to present environmental Vibrio that share some genome sequence homology with the EMS strains. Shrimp infected with only the Ta Mai strain were measured to have between 10,000 and 100,000 cfu per shrimp (A3 &A4) and those pretreated with A603 were measured to have 100-1000× fold fewer Ta Mai strain (B3 & B4). The colonization of shrimp by A603 was measured to be low, between 10-100 per shrimp or per 1 ml water.

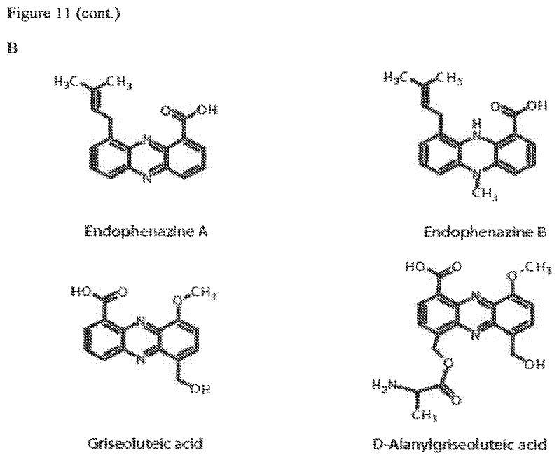

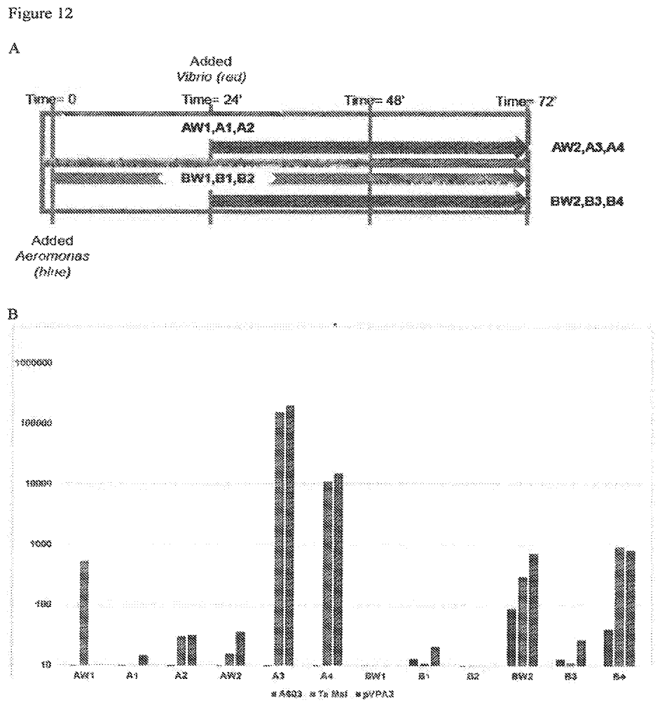

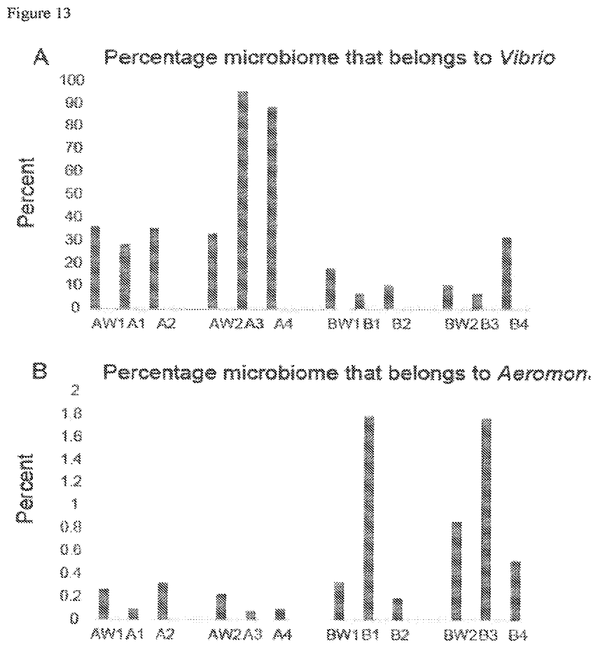

The sequence data for each sample was utilized for additional analysis of the complete microbiome using MG-RAST pipeline (FIG. 13). This analysis used provides statistical information about the abundance of the bacteria present in each water or shrimp sample and can predict an estimate of species diversity. Uninfected shrimp (A1 & A2) were measured to have between 84 and 177 species that were represented by 28-40% Vibrio. These Vibrio are not EMS strains and likely represent common environmental and nonpathogenic species that are endemic to seawater and invertebrates. Shrimp infected with EMS strains for 48 hours (A3 & A4) had a significantly reduced diversity of less than 5 species with EMS strains representing >80% of the microbiome. This collapse of diversity is likely due a significant increase of EMS strains in infected shrimp relative to the native microbiome however it is possible that EMS Vibrio strains either kill other bacteria or elicit a significant innate immune response that has a collateral effect on the native microbiome. Adding Aeromonas A603 to shrimp reduced the species diversity slightly to between 38 and 80 per shrimp with a significant reduction of both environmental (B1 & B2) and environmental EMS Vibrio (B3 & B4) while Aeromonas was calculated to comprise only ˜2% of the microbiome (FIG. 13). This indicates that adding A603 to water results in low level colonization that has minimal impact of the microbiome except for reduction of Vibrio bacteria and prevention of the colonization and explosive expansion of EMS strains in shrimp. In order to examine the role of T6SS in protection, shrimp were infected with EMS strain Ta Mai with and without pre-inoculation of strains A603 and A603 ΔvipA (FIG. 14). In this experiment EMS strain Ta Mai could be selected and enumerated from each shrimp using a streptomycin resistance marker. Using groups of between 26-37 shrimp, both Ta Mai and two other observed abundant bacteria (including Stenotrophomonas) could be measured after treatment. Less than 10 percent of shrimp infected were found to have any detectable level of EMS strain when pretreated with A603 and this level increased to 28% of those pretreated with A603 ΔvipA and more than 70% of those not pretreated. This suggests that A603 defective in T6SS can still provide some protection, albeit reduced, within the infection window. T6SS+ confers the strongest protection from EMS colonization in small shrimp. There was a negative correlation between isolate B (Stenotrophomonas) and EMS colonization and this is consistent with the collapse of the microbiome measured using deep sequencing in FIG. 13C.

### Example 5: The Transcriptomes of Both A603 and Ta Mai Show Both the Phenazine Molecule and T6SS are Antagonistic to AHPNS Strains

The transcriptomes of both A603 and Ta Mai strains were analyzed in order to determine interbacterial antagonism and induced responses in both bacteria when co-cultured. The A603 transcriptome was not impacted significantly within its ˜4300 genes except for a handful of genes within a few operons. All three tetrathionate reducate subunit genes (ttrABC) are upregulated 100-500 fold. These encode a membrane bound complex that reduces tetrathionate to thiosulfate. Concomitantly, a separate operon that encodes a lactate permease and utilization contains the four most downregulated genes (˜20-fold). In Citrobacter freundi, lactate was shown to be a poor electron donor to tetrathionate reduction which may explain this strategy. The dynamic resolution provided in this analyses captures other subtle transcriptional responses likely due to the availability of oxygen, metabolites, or other resources in the co-culture but no indication of stress-related pathways are apparent. This is supported by the absence of killing by A603 Δorf8 ΔvipA by all tested Vibrio species, including a well-characterized V. cholerae isolate with an active T6SS that is significantly bactericidal to many gammaproteobacteria.

In A603, the most highly expressed gene encodes a 71 amino acid protein that is nearly identical to the H. pylori HP 1242 protein. The solved structure of this small protein is composed of three (3-helices and folds in a coiled-coil-like conformation. Both proteins possess a domain of unknown function (DUF465 family) and though this is the most abundantly expressed transcript in this strain, its role is unclear. The remaining four most highly expressed genes encode other proteins, some found to be highly expressed in other gram negative bacteria; these include a S1-like cold shock domain protein, an acyl carrier protein, a FimA-like pilin, and a porin/adhesion. Expanding this list to include the highest 2% of expressed A603 genes in both mono- and co-cultures, it was found that the majority have roles in translation, arginine dihydrolase, ATP synthesis, and other key genes that share common function are related to T6SS or phenazine biosynthesis.

Because AHPNS strains are killed within 120 minutes by A603, the transcriptomes were measured 45 minutes post-incubation to capture responses prior to cell death. In contrast to A603, the Ta Mai transcriptome is significantly altered when co-cultured and the most significantly up-regulated genes are correlated to stress, oxidative damage, and DNA repair. The genes upregulated and downregulated more than 3-fold were extracted and their corresponding GO annotations and abundance were used to extract enrichment of biological processes and pathways using hypergeometric tests. All genes with measured expression were also analyzed using Gene Set Enrichment Analysis (GSEA). Both analyses identified the same A603-induced cell stress pathways in Ta Mai including the DNA repair, SOS response, and oxidation-reduction. These processes were significantly induced when co-cultured with A603 strains operative for T6SS and phenazine molecule biosynthesis. Curiously, pathogenesis was also identified as a significant process as genes that encode Type III secretion and the PirAB toxin components are significantly induced. These genes would normally be induced during infection in a host and may reflect a response initiated by stress mimetic to that of an immune response. A spermidine transmembrane transporter activity is also identified as a key process and the only pathway deemed significant in all co-incubations with A603, regardless of T6SS and the phenazine molecule. The arginine hydrolase pathway is ubiquitously highly expressed in A603 and the polyamine putrescine is a key product of this pathway in other bacteria. Interestingly, different polyamines are produced by both fish and invertebrate tissue decay and to accumulate especially in shrimp hepatopancreatic tissue from fish based feed with no adverse effects. Polyamines are an attractant for some bacteria like Pseudomonas but the role of these in Vibrio chemotaxis is not known. Exogenous polyamines produced by eukaryotes and prokaryotes are shown to enhance biofilm production in V. cholerae. These transcriptional insights may provide clues about how these bacteria interact and may even identify candidate attractant molecules.

Two separate operons were upregulated in Ta Mai more than 1000-fold. GO annotations were not assigned to these genes thus they were not included in the hypergeometric and GSEA analyses. One gene (TMChrII_2378) encodes a protein with high structural conservation to heme monooxgenase and the other (TMChrI_2040) a dioxygenase that closely resembles a bleomycin resistance proteins. A second dioxygenase (TMChr1_3001) is also among the top induced genes. This family of dioxygenase proteins includes those that can bind and in some cases hydrolyze planar and aromatic antibacterial molecules including phenazines. Though amino acid sequence similarity is poor, the predicted structure of TMChr1_2040 is very similar to EphR, a protein that loosely binds phenazine in producer strains and confers resistance by binding molecules until export. To test whether these proteins are operative in resistance, both were cloned and expressed into the V. cholerae O1 strain, H1, a strain very sensitive to the A603 phenazine. Only TMChr1_2040 is shown to confer partial resistance, demonstrating its role is likely similar to other EphR proteins.

### CONCLUSION

Killing assays, genetic approaches and the completed genome of Aeromonas hydrophila A603 were used to identify the T6SS and phenazine biosynthesis operons and determined both are operative, distinct antibacterial mechanisms. Both kill or inhibit the growth of all tested bacteria of the genus Vibrio, including EMS strains isolated from infected and diseased shrimp sourced from Thailand. This antibacterial activity is significantly robust in vitro when large numbers of both predatory A603 and prey Vibrio are incubated together, enough that 10-100 million bacteria are killed in a couple hours. It is worth noting that spontaneous T6SS resistance has yet to be found in any T6SS susceptible gram-negative strain to its competent “predator” strain.

When shrimp were infected with A603 by inoculating environmental water, a low level colonization could be measured with a small effect on the natural shrimp microbiome. In contrast, shrimp infected with EMS-related Vibrio strains exhibited significant dysbiosis with an exponential increase of Vibrio and significant reduction of bacterial diversity. Shrimp pretreated with A603 T6SS+ were largely protected from colonization by EMS and dysbiosis. This protection was significantly reduced by using an A603 T6SS mutant (A603 ΔvipA) suggesting T6SS is a key component of the probiotic effect. When shrimp are treated with EMS, death occurs >80% within one week and with A603 or A603+ EMS mortality was never observed to be more frequent than uninfected (<10% per week, data not shown). These observations suggest A603 is competent for colonizing shrimp via natural uptake from environmental water and that this colonization is not harmful to shrimp or disruptive to the natural shrimp microbiome. Furthermore this provides an advantage to shrimp in that it protects from the colonization by pathogenic strains of Vibrio parahaemolyticus known to cause EMS.

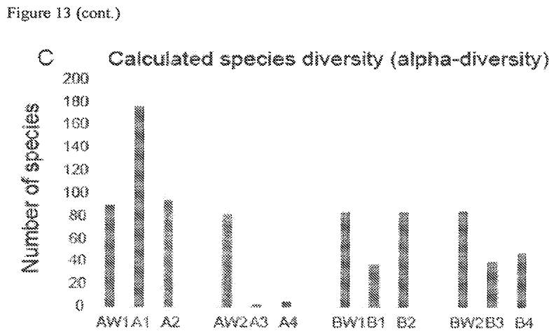

The bacterial inoculum (105-6 cfu/ml) used for protection in these small scale shrimp challenges can easily be scaled up for large tanks or ponds (FIG. 16). A spontaneous streptomycin resistance allele previously selected in A603 can be used for detecting or counting bacteria in shrimp or environmental water and the genome provides information for primers if PCR or qPCR is employed. A603 can be cultured in simple bacterial media between 25° C. and 37° C. and grows quickly to high density. 5-10 liters of A603 growth in a bioreactor could achieve this inoculum for a pond within a day or two at minimal cost. A short term goal is to characterize the regulation of T6SS and phenazine and to engineer the operons to be constitutively upregulated or easily inducible when shrimp and tanks or ponds are treated.

### Additional Data

The micriobiome of AHPND-infected shrimp shows a modest 3-fold increase of bacteria in the Vibrionaceae 72 hours post-infection when compared to DNA reads that are map to about 80 other bacterial families. The increase of mapped DNA to Vibrionaceae in the shrimp body is more than that measured in the hepatopancreas.

RNA extracted and sequenced from the same shrimp shows a significant increase of recovered RNA from the shrimp body, but not the hepatopancreatic tissue. This result suggests bacteria in the Vibrionaceae are much more metabolically active in infected shrimp and that colonization is not specific to the hepatopancreatic tissue, which is where most disease tissue damage has been observed for this bacterial disease.

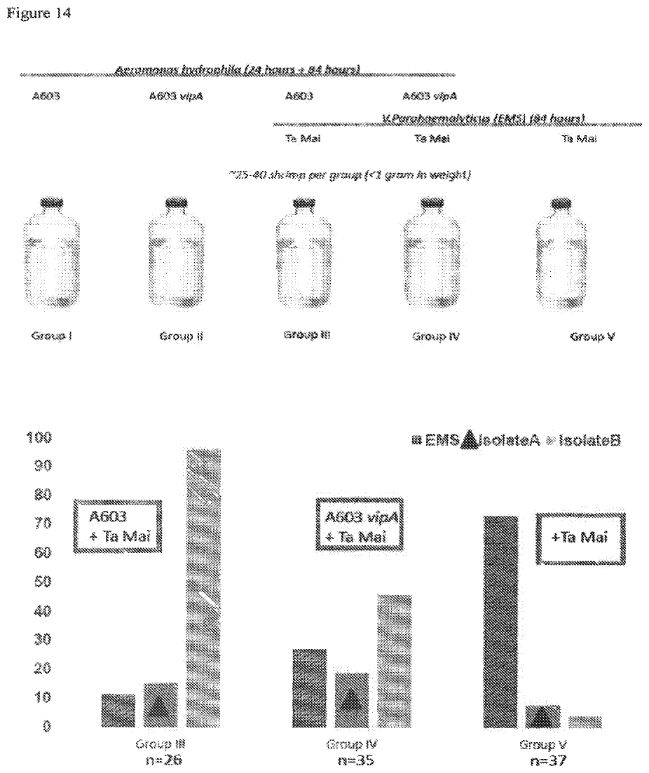

Furthermore, DNA from live or dead cells or that which has been released into the environment cannot be distinguished, but RNA is easily degraded and unstable when released from dead cells. As bacterial chromosomal DNA usually exists as a single copy per cell and the abundance of RNA transcripts per gene is found to vary from less than one to over 50,000 for ribosomal RNA. The greater abundance for RNA suggests these bacteria are alive and metabolically active (FIG. 17).

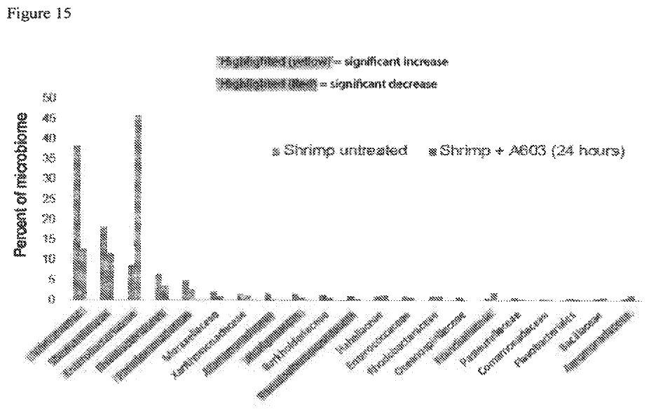

Transcriptome (RNA sequencing) of the shrimp body is the best indicator of the increased bacterial load of Vibrionaceae. Using this metric, it is shown that a 24-fold increase of Vibrionaceae bacteria in infected shrimp after 72 hours. The only other bacteria family that appears to track closely with Vibrio-related species in abundance are those in the Aeromonadaceae. By sequence, these Aeromonas bacteria are not the A603 strain and do not appear to eliminate Vibrio from shrimp, but the observation indicates that AHPND-infected shrimp may become more vulnerable to colonization by Aeromonads (FIG. 18).

Using the transcriptome data, shrimp pretreated with A603 have 3-fold fewer Vibrionaceae and 8-fold fewer Aeromonadaceae after 72 hours. There are three shrimp per group in the experiment and each plot point represents the average abundance of bacterial family as in FIGS. 17 and 18.

The data from these independent groups shows the same coincidental tracking of Vibrionaceae with Aeromonadaceae, but in the first groups (FIGS. 17 and 18), the Aeromonads are environmental. When A603-pretreated shrimp are challenged with AHPND V. parahaemolyticus the burden of Vibrionaceae and Aeromonadaceae is reduced 10× and 97×, respectively. There are several possible models to explain the selective co-targeting of these specific bacterial families but all conclude that A603 eliminates both AHPND and other Vibrio strains from shrimp. A603 may also colonize in a Vibrio-dependent manner as A603-treated shrimp are found to have an only minute bacterial burden of A603. Because bacteria that belong to the Vibrionaceae and environmental Aeromonaceae are found to correlate in abundance, targeted clearance of these by A603 may occur by a common mechanism or either bacteria in the Vibrionaceae and Aeromonadaceae contribute to the growth of the other.

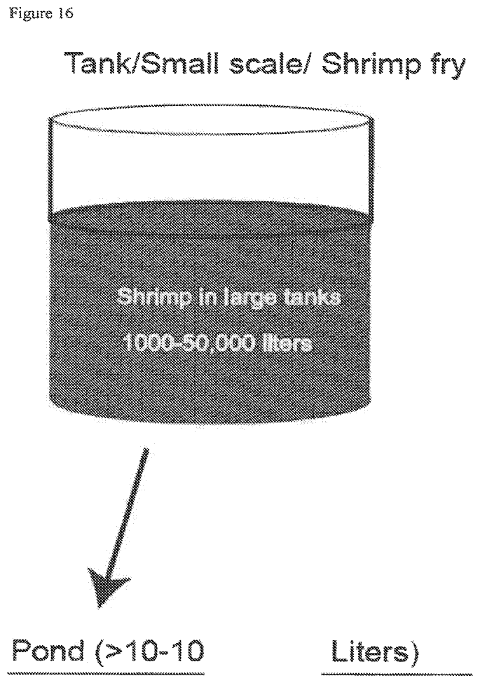

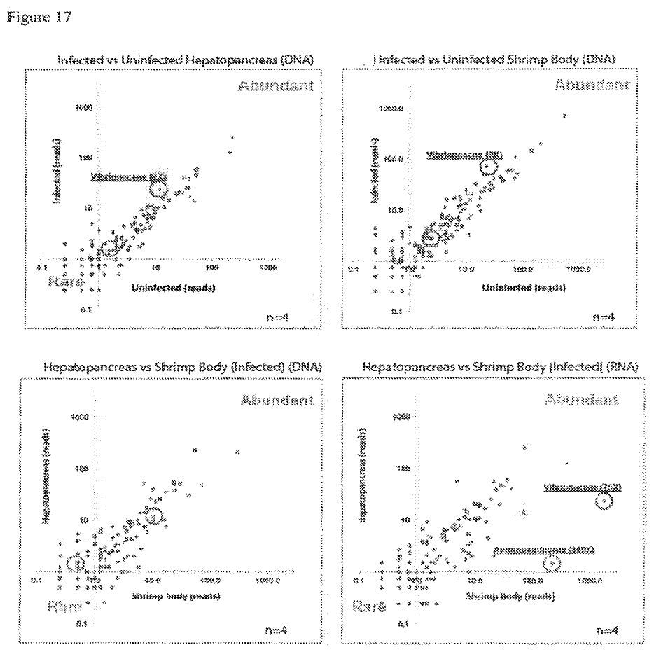

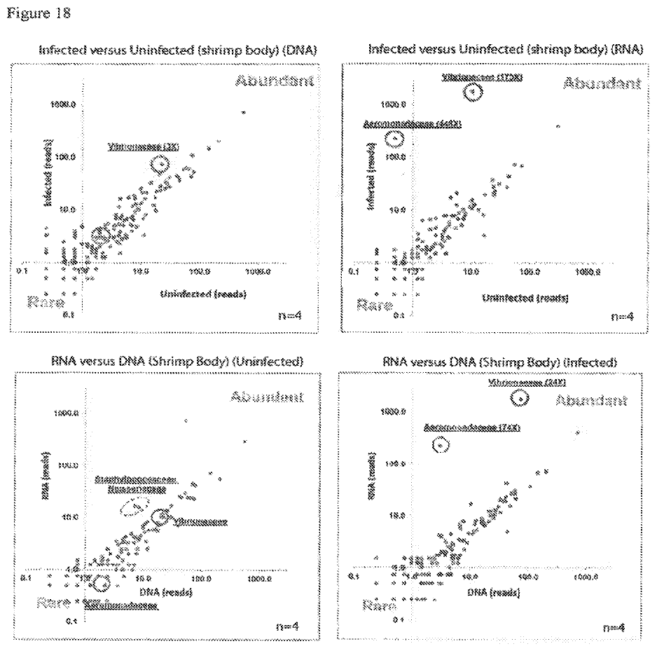

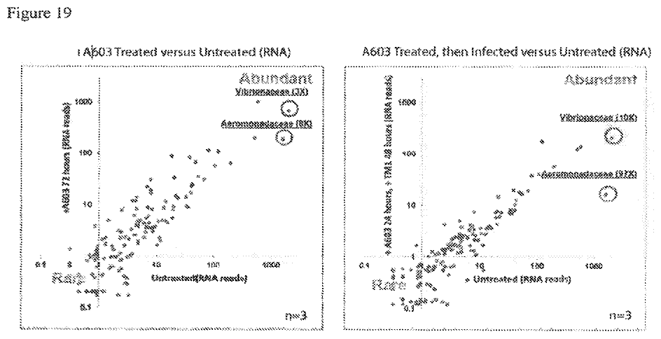

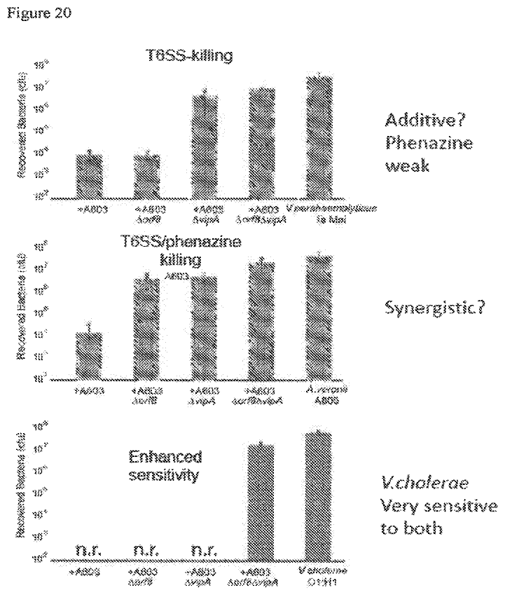

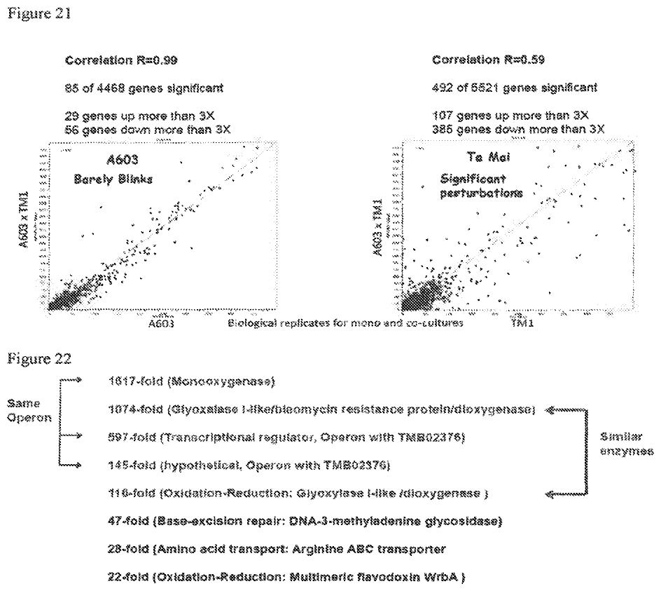

Because other species, strains, and serotypes of Aeromonas and Vibrio, in addition to V. parahaemolyticus, cause disease in shellfish, fish, and humans, A603 could be a used as preventive treatment for a number of pathogens. These include, but are not limited to, V. vulnificus, V. harveyi, V. cholerae, V. aliginolyticus, A. hydrophila, and A. media. Every representative isolate of Vibrio and Aeromonas species in this list of six have been shown to be killed by A603. A603 does appear to have either minor or no impact on most bacteria families in shrimp confirming the antibacterial activity is targeted to bacteria families that includes many species that cause disease (FIG. 19).

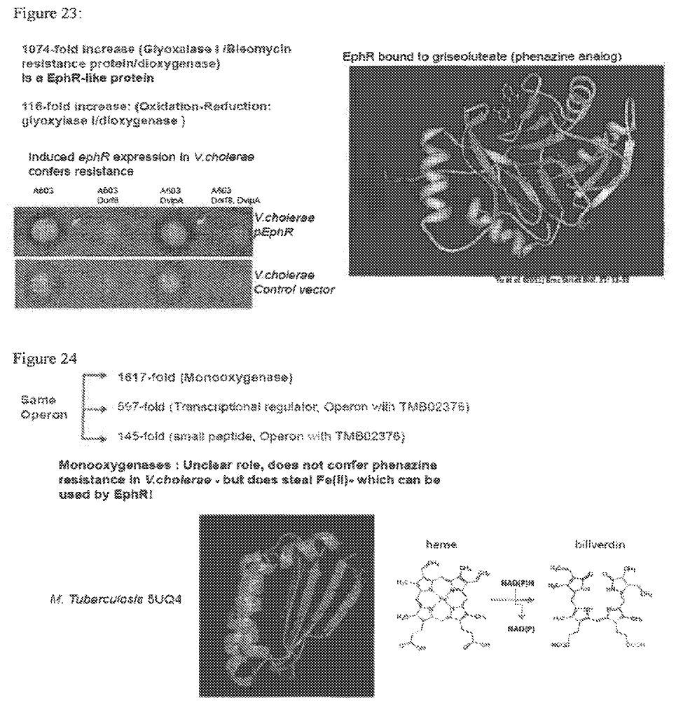

FIG. 25 shows whether A603 will work as an antibacterial in the environment or host. Shrimp were pretreated by adding A603 to water 10E5/ml cfu. V. parahaemolyticus (EMS strain, StrR) was added after 24 hours. Selection and enumeration were performed by using the streptomycin marker. The following was observed: 1) water treated with A603 protects shrimp from being colonized by EMS strain Ta Mai, 2) protection is largely reduced but not eliminated when a A603 T6SS mutant is used, thus T6SS is a key antibacterial mechanism in vivo, and 3) other strains in shrimp are negatively correlated with EMS colonization—maybe EMS antagonizes other bacteria.

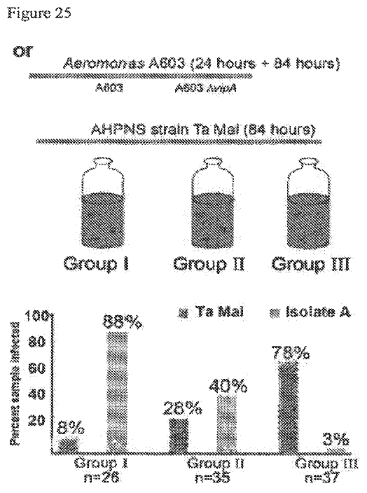

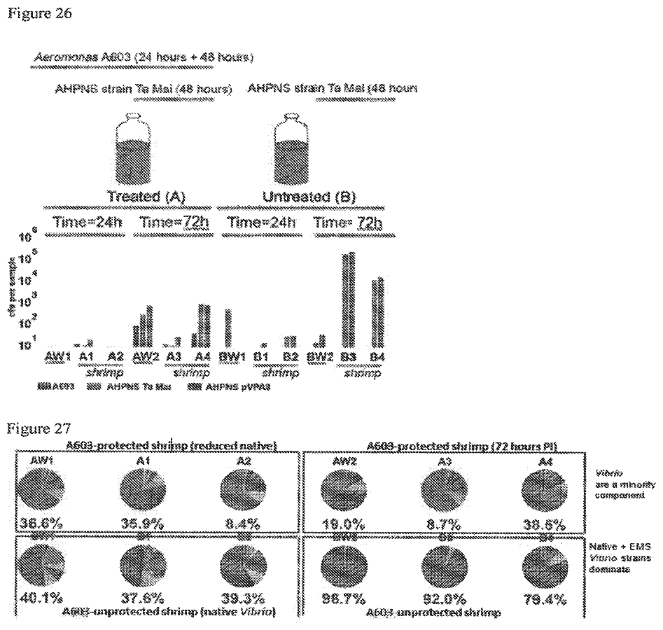

Protection in shrimp was qualified using NGS sequencing. Protection in A603 can be measured by a marked quantitative reduction in V. parahaemolyticus (or Vibrio) in shrimp. Bacteria extracted from shrimp were sequenced using NGS (Illumina) and A603 and V. parahaemolyticus were quantified against an added DNA standard. FIG. 26 shows that shrimp pretreated with A603 are colonized 100-10,000× less than those untreated. A603 is not a significant colonizer after 72 hours, even when added to water at 10E5/ml cfu. A603-treated shrimp have reduced colonization, not only by added EMS strains but also by endemic, environmental Vibrio strains. Bacteria extracted from shrimp were sequenced using NGS (Illumina) and mapped by bacteria family using MG-RAST. Is was observed that Vibrio naturally make up part of the shrimp microbiome. The V. parahaemolyticus in EMS infected shrimp can be >90% of the microbiome. (Vibrionaceae reflected in percentages) (FIGS. 26 and 27).

The following table shows upregulated pathways in V. parahaemolyticus EMS strain depend on T6SS and phenazine.

TABLE 1
Upregulated pathways in V. parahaemolyticus EMS strain depend on T6SS and Phenazine

| +A603 | +A603 del_orf8 | +A603 del_vipA | +A603 double mutant |
|---|---|---|---|
| T6SS+ | T6SS+ | No T6SS | No T6SS |
| Phenazine+ | No Phenazine | Phenazine+ | No Phenazine |
| DNA repair (8/29) | DNA Repair (3/29) | DNA Repair (7/29) | DNA repair (4/29) |
| SOS Response (4/9) | | SOS response (4/9) | |
| Spermidine | Spermidine | Spermidine | Spermidine |
| transport (2/3) | transport (2/3) | Transport (2/3) | Transport (2/3) |
| Ox/Redox | Ox/Redox Processes (18/276) | DNA recombination | Negative Regulation |
| Processes (16/276) | | (3/30) | Of Cell Division (1/1) |
| Pathogenesis (5/11) | Bacterial-type flagellum | | |
| | Organization (3/15) | | |
| Regulation of Protein | Response to Stress (3/16) | | |
| Secretion (3/6) | | | |

*RNA seq analysis (CLC-Bio Workbench 8.1) and GO analysis (Blast2Go Pro)
Reduction in key pathways (compared to WT)

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating or preventing acute hepatopancreatic necrosis syndrome (AHPNS) or early mortality syndrome (EMS) in cultured crustaceans comprising administering Aeromonas hydrophila A603 bacteria to environmental waters comprising the cultured crustaceans.

2. A method of inhibiting the growth of pathogenic bacteria in cultured crustaceans comprising administering Aeromonas hydrophila A603 bacteria to environmental waters comprising the cultured crustaceans.

3. The method of claim 2, wherein the pathogenic bacteria are Vibrio bacteria.

4. The method of claim 3, wherein the Vibrio bacteria are V. cholera, V. vulnificus, V. harveyi, V. cholerae, V. aliginolyticus. A. hydrophila, or A. media.

5. The method of claim 3, wherein the Vibrio bacteria are V. parahaemolyticus.

6. The method of claim 2, wherein the pathogenic bacteria are A. hydrophila.

7. The method of claim 2, wherein the pathogenic bacteria are A. media.

8. The method of any one of claims 2 to 7, wherein the pathogenic bacteria are antibiotic resistant.

9. The method of any one of claims 2 to 8, wherein the pathogenic bacteria is associated with acute hepatopancreatic necrosis syndrome (AHPNS) or early mortality syndrome (EMS).

10. The method of any one of claims 2 to 8, wherein the pathogenic bacteria is not associated with acute hepatopancreatic necrosis syndrome (AHPNS) or early mortality syndrome (EMS).

11. A method of treating or preventing bacterial infection in cultured crustaceans comprising administering Aeromonas hydrophila A603 bacteria to environmental waters comprising the cultured crustaceans.

12. The method of claim 11, wherein the bacterial infection is caused by Vibrio bacteria.

13. The method of claim 12, wherein the Vibrio bacteria are V. cholera, V. vulnificus, V. harveyi, V. cholerae, V. aliginolyticus. A. hydrophila, or A. media.

14. The method of claim 12, wherein the Vibrio bacteria are V. parahaemolyticus.

15. The method of claim 11, wherein the bacterial infection is caused by A. hydrophila, A. caviae, A. sobria, or A. media.

16. The method of claim 11, wherein the bacterial infection is caused by A. hydrophila or A. media.

17. The method of any one of claims 11 to 16, wherein the bacterial infection is caused by pathogenic bacteria that are antibiotic resistant.

18. The method of any one of claims 11 to 17, wherein the pathogenic bacteria causing the bacterial infection is associated with acute hepatopancreatic necrosis syndrome (AHPNS) or early mortality syndrome (EMS).

19. The method of any one of claims 11 to 17, wherein the pathogenic bacteria causing the bacterial infection is not associated with acute hepatopancreatic necrosis syndrome (AHPNS) or early mortality syndrome (EMS).

20. A method of overcoming or inhibiting antibiotic resistance in cultured crustaceans comprising administering Aeromonas hydrophila A603 bacteria to environmental waters comprising the cultured crustaceans.

21. The method of any one of claims 1 to 20, wherein the cultured crustaceans are shrimp.

22. The method of claim 21, wherein the shrimp are Litopenaeus vannamei shrimp.

23. The method of any one the preceding claims, wherein the environmental water is seawater.

24. The method of any one of claims 1 to 22, wherein the environmental water is brackish water.

25. The method of any one of the preceding claims, wherein the method further comprises contacting Aeromonas hydrophila A603 bacteria with an agent that increases the expression of T6SS proteins prior to administering the Aeromonas hydrophila A603 bacteria to the environmental waters comprising the cultured crustaceans.

26. The method of claim 25, wherein the agent is an expression vector.

27. The method of claim 26, wherein the expression vector is an expression vector encoding for a T6SS effector protein or a T6SS machinery protein.

28. The method of any one of the preceding claims, wherein the method further comprises contacting the Aeromonas hydrophila A603 bacteria with an agent that activates phenazine biosynthesis in Aeromonas hydrophila A603 prior to administering the Aeromonas hydrophila A603 bacteria to the environmental waters comprising the cultured crustaceans.

29. The method of claim 28, wherein agent is a AHL molecule.

30. The method of claim 28, wherein the agent is a PhzR protein.

31. The method of claim 28, wherein the agent is an expression vector.

32. The method of claim 31, wherein the agent is an expression vector comprising a gene or a portion of a gene in the phenazine operon.

33. The method of any one of the preceding claims, wherein Aeromonas hydrophila A603 is administered to the environmental water comprising the cultured crustaceans conjointly with a phenazine, a phenazine precursor, or a phenazine derivative.

34. The method of claim 33, wherein the phenazine is pyocyanin.

35. The method of any one of the preceding claims, wherein Aeromonas hydrophila A603 is administered to the environmental waters comprising the cultured crustaceans conjointly with an antibiotic.

36. The method of claim 35, wherein the antibiotic is oxytetracycline, florfenicol, sarafloxacin, enrofloxacin, chlortetracycline, quinolones, ciprofloxacin, norfloxacin, oxolinic acid, perfloxacin, sulfamethazine, gentamicin, or tiamulin.

37. A composition comprising Aeromonas hydrophila A603.

38. The composition of claim 37, wherein the composition further comprises a phenazine.

39. The composition of claim 38, wherein the phenazine is pyocyanin.

40. The composition of any one of claims 37 to 39, wherein the composition further comprises an AHL.

41. The composition of any one of claims 37 to 40, wherein the composition further comprises a PhzR protein.

42. The composition of any one of claims 37 to 41, wherein the composition further comprises an antibiotic.

43. The composition of any one of claims 37 to 42, wherein the composition is a probiotic.

44. The composition of any one of claims 37 to 42, wherein the composition is aquaculture feed.
